(19) United States
(12) Patent Application Publication
(10) Pub. No.: US 2025/0079025 A1
(43) Pub. Date: Mar. 6, 2025

(54) PATHOGEN TEST DEVICE

(71) Applicant: Samantha Froebe, Eudora, KS (US)

(72) Inventor: Samantha Froebe, Eudora, KS (US)

(21) Appl. No.: 18/241,173

(22) Filed: Aug. 31, 2023

(51) Int. Cl. G16H 70/60; G16H 50/80

(52) U.S. Cl. CPC G16H 70/60; G16H 50/80

(57) ABSTRACT

A pathogen test device, including a center body to facilitate gripping thereof, a swab connected to at least a portion of a first end of the center body to absorb at least one fluid therein in response to contact with the at least one fluid, a testing container detachably connected to at least a portion of a second end of the center body to store a testing liquid therein, and a test display disposed on at least a portion of the center body to display a test result identifying a presence of at least one of COVID-19, strep, and control in response to inserting the swab with the at least one fluid into the testing container.

### BACKGROUND

### 1. Field

The present general inventive concept relates generally to pathogen testing, and particularly, to a pathogen test device.

### 2. Description of the Related Art

Since late 2019, coronavirus disease 2019 (COVID-19) has created a worldwide pandemic. For people that develop symptoms from the disease, most are respiratory complications, such as difficulty breathing, coughing, and/or headache. Unfortunately, these types of symptoms are similar to many other infections. Additionally, being infected with other types of pathogens can complicate identification of diseases.

It is common for some people to also be infected with streptococcal pharyngitis (a.k.a., strep throat) while infected with COVID-19 due to frequent coughing which can eject many pathogens. Unfortunately, confirmation of strep throat requires visiting a doctor's office to obtain results.

Therefore, there is a need for a pathogen test device that can quickly identify multiple different pathogens.

### SUMMARY

The present general inventive concept provides a pathogen test device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a pathogen test device, including a center body to facilitate gripping thereof, a swab connected to at least a portion of a first end of the center body to absorb at least one fluid therein in response to contact with the at least one fluid, a testing container detachably connected to at least a portion of a second end of the center body to store a testing liquid therein, and a test display disposed on at least a portion of the center body to display a test result identifying a presence of at least one of COVID-19, strep, and control in response to inserting the swab with the at least one fluid into the testing container.

The pathogen test device may further include a rod disposed on the first end of the center body to connect the swab to the center body.

The rod may absorb and transfer the at least one fluid and the testing liquid on the swab toward the center body while the swab is inserted within the testing container.

The test display may indicate the test result after a predetermined period of time.

The test display may identify COVID-19 and strep in response to a simultaneous infection.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates an isometric view of a pathogen test device, according to an exemplary embodiment of the present general inventive concept; and

FIG. 2 illustrates a side perspective view of the pathogen test device, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Pathogen Test Device 100
Center Body 110
Swab 120
Rod 130
Testing Container 140
Test Display 150
Swab Cap 160

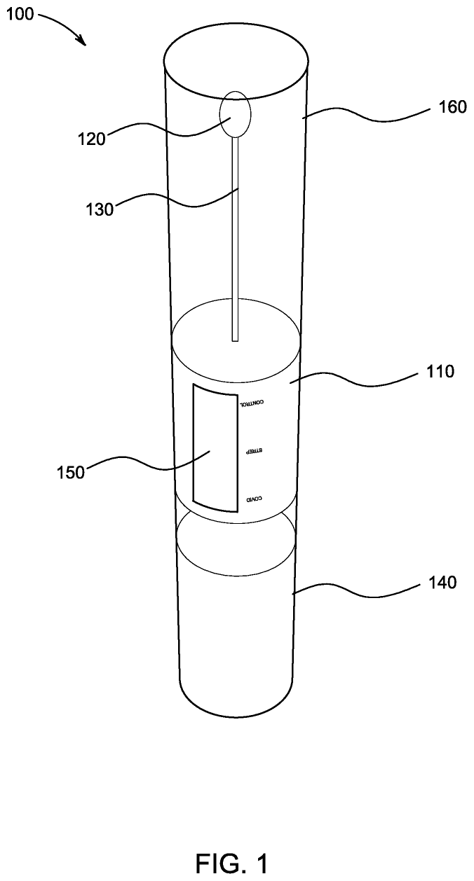

FIG. 1 illustrates an isometric view of a pathogen test device 100, according to an exemplary embodiment of the present general inventive concept.

The pathogen test device 100 may be constructed from at least one of metal, plastic, wood, cloth, glass, and rubber, etc., but is not limited thereto.

The pathogen test device 100 may include a center body 110, a swab 120, a rod 130, a testing container 140, a test display 150, and a swab cap 160, but is not limited thereto.

Referring to FIG. 1, the center body 110 is illustrated to have a cylindrical shape. However, the center body 110 may be rectangular, circular, triangular, pentagonal, hexagonal, heptagonal, octagonal, or any other shape known to one of ordinary skill in the art, but is not limited thereto.

The center body 110 may be used as a handle to facilitate gripping thereof. Moreover, the center body 110 may have a textured surface on at least a portion of an outer surface thereof. For example, the center body 110 may have a sandpaper surface, a knurled surface, a ribbed surface, and/or any combination thereof.

The swab 120 may include an absorbent cloth, cotton, and/or any other type of cloth, but is not limited thereto.

The swab 120 may be connected to at least a portion of the center body 110. The swab 120 may absorb at least one fluid (e.g., water, mucus, etc.) therein in response to contact with the at least one fluid. For example, the swab 120 may absorb saliva in response to being inserted in a mouth and/or swabbing against a cheek of a user. Therefore, the swab 120 may facilitate extraction of the at least one fluid.

The rod 130 may have an elongate shape. The rod 130 may be disposed on a first end of the center body away from the center body 110 with respect to a direction. The rod 130 may connect the swab 120 to the center body 110. As such, the swab 120 may be disposed at a distance away from the center body 110 equivalent to a length of the rod 130. The rod 130 may be preconfigured to absorb and/or transfer the at least one fluid on the swab 120 toward the center body 110.

The testing container 140 may be detachably connected (i.e., snap, screwed, clipped) to at least a portion of a second end of the center body 110 opposite with respect to the first end of the center body 110. The testing container 140 may store a testing liquid (e.g., sodium azide) therein.

The test display 150 may be disposed on at least a portion of the center body 110. The test display 150 may be preconfigured to identify a plurality of infected conditions. More specifically, the test display 150 may be preconfigured to identify a presence of COVID-19, strep, and/or control (i.e., uninfected). In other words, the test display 150 may display to the user which type of pathogen has infected the body of the user. The test display 150 may display a result after inserting the swab 120 into the testing container 140 for a predetermined period of time (e.g., fifteen minutes).

The swab cap 160 may be detachably connected (i.e., snap, screwed, clipped) to at least a portion of the first end of the center body 110. The swab cap 160 may cover the swab 120 and/or the rod 130 therein.

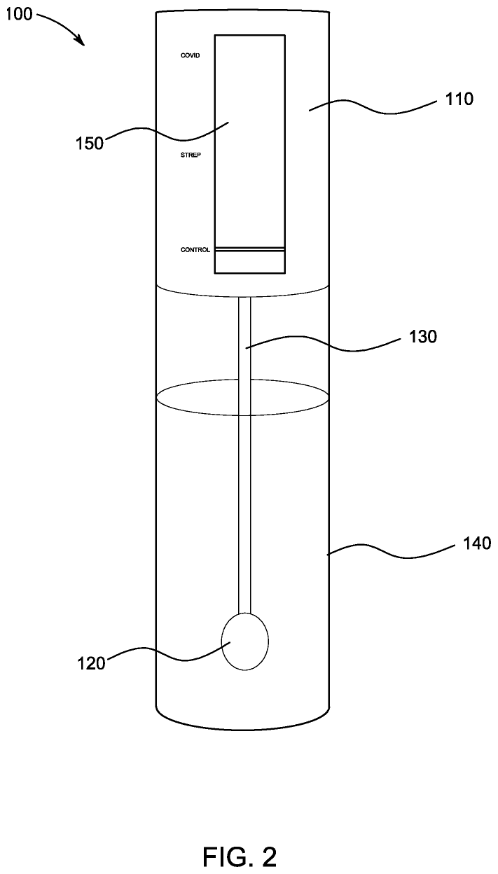

FIG. 2 illustrates a side perspective view of the pathogen test device 100, according to an exemplary embodiment of the present general inventive concept.

During use, the swab cap 160 may be removed. The swab 120 may be swabbed within the mouth of the user and/or against a cheek of the user to obtain a fluid sample. The testing container 140 may be removed. Subsequently, the swab 120 and/or the rod 130 may be disposed (i.e., inserted) into the testing container 140 and/or within the testing liquid. The rod 130 may transfer the fluid sample and/or the testing liquid toward the center body 110. Accordingly, the test display 150 may display a test result, such as COVID-19, strep, and/or control. Also, the test display 150 may simultaneously display COVID-19 and strep, which indicates simultaneous infection. Furthermore, an entire testing process may be rapid (e.g., less than fifteen minutes) to obtain the test result.

Therefore, the pathogen test device 100 may provide quick and/or accurate results. Also, the pathogen test device 100 may be a home solution that does not require a doctor's visit to obtain test results.

The present general inventive concept may include a pathogen test device 100, including a center body 110 to facilitate gripping thereof, a swab 120 connected to at least a portion of a first end of the center body 110 to absorb at least one fluid therein in response to contact with the at least one fluid, a testing container 140 detachably connected to at least a portion of a second end of the center body 110 to store a testing liquid therein, and a test display 150 disposed on at least a portion of the center body 110 to display a test result identifying a presence of at least one of COVID-19, strep, and control in response to inserting the swab 120 with the at least one fluid into the testing container 140.

The pathogen test device 100 may further include a rod 130 disposed on the first end of the center body 110 to connect the swab 120 to the center body 110.

The rod 130 may absorb and transfer the at least one fluid and the testing liquid on the swab 120 toward the center body 110 while the swab 120 is inserted within the testing container 140.

The test display 150 may indicate the test result after a predetermined period of time.

The test display 150 may identify COVID-19 and strep in response to a simultaneous infection.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A pathogen test device, comprising:
a center body to facilitate gripping thereof;
a swab connected to at least a portion of a first end of the center body to absorb at least one fluid therein in response to contact with the at least one fluid;
a testing container detachably connected to at least a portion of a second end of the center body to store a testing liquid therein; and
a test display disposed on at least a portion of the center body to display a test result identifying a presence of at least one of COVID-19, strep, and control in response to inserting the swab with the at least one fluid into the testing container.

2. The pathogen test device of claim 1, further comprising:
a rod disposed on the first end of the center body to connect the swab to the center body.

3. The pathogen test device of claim 2, wherein the rod absorbs and transfers the at least one fluid and the testing liquid on the swab toward the center body while the swab is inserted within the testing container.

4. The pathogen test device of claim 1, wherein the test display indicates the test result after a predetermined period of time.

5. The pathogen test device of claim 1, wherein the test display identifies COVID-19 and strep in response to a simultaneous infection.
